(19) United States
(12) Patent Application Publication
(10) Pub. No.: US 2024/0381867 A1
(43) Pub. Date: Nov. 21, 2024

(54) BIO-OIL FRACTIONS COMPOSITION DERIVED FROM BIO-OIL

(71) Applicant: Suzano S.A., Salvador (BR)

(72) Inventors: Danilo Ribeiro DE LIMA, São José dos Campos (BR); Marcus Paulo MARTINS, Americana (BR); Matheus Antunes GUIMARÃES, Aracruz (BR); HELOISA OGUSHI ROMEIRO RAMIRES, Aracruz (BR); EVERTON PIRES SOLIMAN, ltapetininga (BR); SAULO DE MELO XAVIER SILVA, ltapetininga (BR); Edival Angelo Valverde ZAUZA, ltapetininga (BR)

(73) Assignee: Suzano S.A., Salvador, SA (BR)

(21) Appl. No.: 18/686,345

(22) PCT Filed: Aug. 25, 2022
(86) PCT No.: PCT/BR2022/050333

§ 371 Date: Feb. 23, 2024

Related U.S. Application Data
(60) Provisional application No. 63/237,034, filed on Aug. 25, 2021.

(51) Int. Cl. A01N 37/02; A01N 43/90; A01P 7/04; A01P 13/00; C07G 1/00

(52) U.S. Cl. CPC A01N 37/02; A01N 43/90; A01P 7/04; A01P 13/00; C07G 1/00

(57) ABSTRACT

The invention is directed to bio-oil fractions compositions comprising anhydro sugars and acetic acid obtained from bio-oils when subjected to polar solvent mixtures, to the process for the preparation of a bio-oil fractions composition, the uses thereof, as well as a desiccant herbicide and a crop insecticide comprising said bio-oil fractions composition.

Further, the invention is also directed to a method for preparing a dispersion composed of an oil phase and a non-oil phase, by contacting a bio-oil with a polar solvent, and to a method for extracting a bio-oil fractions composition and a pyrolytic lignin from the dispersion.

### FIELD OF INVENTION

The invention is directed to bio-oil fractions compositions obtained from bio-oils when subjected to polar solvent mixtures.

### BACKGROUND

Bio-oils may typically be obtained from several lignocellulosic sources. One source of bio-oil is forest biomass, such as hardwood, softwood, grasses and weeds; sugarcane, including bagasse, straw harvests; energy cane as a whole, or stems; and their mixtures. A preferred source to produce the bio-oil used in the present invention is lignocellulosic material obtained from 25% hardwood and 75% softwood. However, other sources may be used. Bio-oils usually contain more than 500 identified components derived from cellulose, hemicellulose, lignin and extractives.

In its typical composition, bio-oil contains water (10-30% wt.) and organic compounds such as carboxylic acids, aldehydes, ketones, anhydro sugars (levoglucosan) and phenolic compounds. Fast pyrolysis of biomass produces typical bio-oil samples with high oxygen content (>30% m/m), high concentration of organic acids and low energy density. Levoglucosan (1,6-Anhydro-beta-D-glucopyranose, CAS 498-07-7) is mainly produced from biomass cellulose pyrolysis, and it is a versatile molecule with a growing market potential.

Typically, the bio-oil of interest for the present invention may have considerable amounts of methylphenol and methoxyphenol isomers, alkylmethoxyphenol isomers, benzenediol isomers such as hydroquinone (1,4-benzenediol), resorcinol (1,3-benzenediol) and catechol (1,2-benzenediol); furfural; cyclic ketones and hydroxypropanones and anhydro sugars such as levoglucosan (approximately 20% wt.) and thermal polymerized and partially depolymerized lignin (approximately 40% wt.). Levoglucosan sugars are ingredients with high added value used in the pharmaceutical industry, flavors and fragrances, further being a precursor to produce solvents and polymers. For example, levoglucosan may be used as a feedstock in the production of (hydroxymethyl) furfural (HMF), dihydrolevoglycosan, 1,6-hexanediol and aprotic solvents.

A fast pyrolysis process is preferably used to obtain a suitable bio-oil for the present invention. In this process, energy in the form of heat is applied, with or without a catalyst, to convert organic matter into building blocks used for producing liquid and biochemical fuels currently produced by the petrochemical industry through different synthetic routes. The pyrolysis process is a thermochemical method that meets environmental requirements and provides one of the most competitive means to obtain combustible material from agricultural, forestry and industrial lignocellulosic materials. In addition, the pyrolysis process adds value to the low-value raw material such as lignocellulosic residues, converting it into biofuels and marketable bioproducts.

Each fraction of biomass (cellulose, hemicellulose and lignin) results in a distinct group of chemical components after thermal depolymerization.

The U.S. Pat. No. 8,083,900 discloses a method to separate water from bio-oil in a partial condenser.

The document of Oasmaa, A., et al. “Controlling the phase stability of biomass fast pyrolysis bio-oil” discloses the phase stability of bio-oil in water and other solvents. Said document mentions the possibility of fractionating the bio-oil by the interpretation of the stability curves, without detailing properties, features, and applications of the bio-oil fractions.

Although the bio-oil and its process of preparation and fractionation is known from the state of the art, it is imperative to develop a process for separating the fractions of the bio-oil with improved features, that is, a process for fractionating the bio-oil into commercially desirable bio-oil fractions, which can then be used in several applications such as desiccants, bio-insecticides, herbicides, polyurethane foams, resins, energy production, among others.

The present invention refers to obtaining enhanced bio-oil fractions of commercial interest. Specifically, the invention provides a process to obtain bio-oil fractions such as aqueous phase rich in levoglucosan and acetic acid, and organic phase rich in aromatic oligomers and polymeric lignin. The present invention also provides the uses of the bio-oil fractions composition.

### BRIEF DESCRIPTION OF THE INVENTION

The present invention is directed to a composition comprising anhydro sugars and acetic acid wherein the weight proportion of anhydro sugars to acetic acid is from 0.8-1.5. In such composition the anhydro sugars are present from 50 g/L-110 g/L and the acetic acid is present from 70 g/L-120 g/L. Preferably, the composition has a proportion of anhydro sugars to acetic acid of 1.0, while having a having a pH from 3-4 and a dynamic viscosity from 2-5 cP. Typically, this is achieved with a 20-40% solids content. Elemental analysis reveals a range of composition of aqueous phase containing chemicals such as levoglucosan and acetic acid of the present invention as having 20% carbon, 10% hydrogen, 0.10% nitrogen, less than 0.03% sulfur and about 70% oxygen.

It is also provided a method for preparing a dispersion composed of an oil phase and a non-oil phase, by contacting a bio-oil with a polar solvent, the method comprising:
a) providing a bio-oil;
b) providing a polar solvent;
c) mixing the bio-oil and the polar solvent for 5-120 minutes.

The present invention also provides a method for extracting a composition from the dispersion, the method further comprising:
d) decanting the mixture of bio-oil and polar solvent for 3-48 hours and/or centrifuging the bio-oil and polar solvent mixture for 1-15 minutes;
e) retrieving the composition comprising anhydro sugars and acetic acid having a proportion of anhydro sugars to acetic acid from 0.8-1.5 in weight from one of the separated phases of the mixture from step d).

Other embodiment of the invention refers to a method for extracting a pyrolytic lignin from the dispersion, the method further comprising:
d) decanting the mixture of bio-oil and polar solvent and/or centrifuging the bio-oil and polar solvent mixture;
e) retrieving the pyrolytic lignin from one of the separated phases of the mixture from step d).

The present invention also provides a process for the preparation of a composition comprising anhydro sugars and acetic acid having a proportion of anhydro sugars to acetic acid from 0.8-1.5 in weight, the process comprising:
a) providing a bio-oil;
b) providing a polar solvent;
c) mixing the bio-oil and polar solvent during 5-120 minutes;
d) decanting the mixture of bio-oil and polar solvent and/or centrifuging the bio-oil and polar solvent mixture;
e) retrieving the composition comprising anhydro sugars and acetic acid having a proportion of anhydro sugars to acetic acid from 0.8-1.5 in weight from one phase of the separated phases of the mixture from step d).

Other embodiment of the invention refers to a pyrolytic lignin comprising phenols and benzaldehydes, an herbicide comprising a bio-oil fractions composition, the use thereof, as well as an insecticide comprising a bio-oil fractions composition.

### BRIEF DESCRIPTION OF THE DRAWINGS

FIG. 1 is a chart showing the percentage of water in the total system based on the water/bio-oil ratio during the fractionation process.

FIG. 2 is a chart showing the desiccation results of the bio-oil fractions composition (BOFC) in different application dosages.

FIG. 3 is a chart showing the desiccation results of the bio-oil fractions composition (BOFC) in different application dosages.

FIG. 4 is a chart showing the use of the bio-oil fractions composition in different aqueous concentrations and compared with a commercial insecticide.

FIG. 5 is a chart showing the effect from the use of a salt as an additive during the bio-oil fractionation.

### DETAILED DESCRIPTION OF THE INVENTION

One embodiment of this invention is directed to a bio-oil fractions composition comprising anhydro sugars and acetic acid wherein the weight proportion of anhydro sugars to acetic acid is from 0.8-1.5. In such composition the anhydro sugars are present from 50 g/L-110 g/L and the acetic acid is present from 70 g/L-120 g/L. Preferably, the composition has a proportion of anhydro sugars to acetic acid of 1.0, while having a pH from 3-4 and a dynamic viscosity from 2-5 cP.

Typically, this is achieved with a 20-40% wt. solids content. A typical Elemental Analysis reveals the present invention's bio-oil fractions composition aqueous phase as having 20% carbon, 10% hydrogen, 0.10% nitrogen, less than 0.03% sulfur and about 70% oxygen.

The fractionation of the bio-oil results in an aqueous phase and an oily phase. As used herein, the term “bio-oil fractions composition” refers to the aqueous phase resulting from the fractionation of the bio-oil and the term “pyrolytic lignin” refers to the oily phase resulting from the fractionation of the bio-oil.

The bio-oil fractions composition aqueous phase further comprises hydroxyacetone, glycolaldehyde and/or benzenediols, preferably from 1-2% wt. of hydroxyacetones, from 1-3% wt. of glycolaldehydes and from 4-6% wt. of benzenediols isomers.

The anhydro sugars of the composition are mainly levoglucosan, although other sugars may be present.

The composition also comprises a polar solvent. Preferably, the polar solvent is selected from the group consisting of water, alcohol, effluents from existing factories and mixtures thereof. If the polar solvent is an alcohol, it is preferably a C₁-C₆ alcohol. Preferably, the alcohol is methanol or ethanol. If the polar solvent is an effluent from existing factories, it is preferably an effluent from the pulp and paper production factory. The use of an effluent from an existing factory as the polar solvent results in an integrated process, wherein the effluent from an existing factory is used as the solvent for the fractionation of the bio-oil.

In addition, the composition may further comprise at least one component as an additive, said component being selected from the group consisting of: a) at least one surfactant; and b) at least one salt.

The salt or surfactant do not necessarily form part of the bio-oil fractions composition but are used for producing the bio-oil fractions compositions. Usually, the process undergoes a separation step, wherein the salt or surfactant provides a better separation or more defined separation phases and some traces may remain in the final composition. Therefore, any salt or surfactant is preferred to produce optimum separation of an oil phase from a non-oil phase (aqueous phase). Adequate surfactants or demulsifiers agents are selected from the group consisting of alkyl benzene sulphonates, methyl methacrylate, kraft lignin, lignosulphonates, cellulose derivatives and mixtures thereof. Also, adequate salts are selected from the group consisting of sodium chloride, calcium chloride, magnesium chloride, phosphates, pyrophosphates, sulfates, sulfides, carbonates, nitrates and mixtures thereof.

One embodiment of the present invention is a process for the preparation of a bio-oil fractions composition comprising anhydro sugars and acetic acid having a proportion of anhydro sugars to acetic acid from 0.8-1.5 in weight comprising:
a) providing a bio-oil;
b) providing a polar solvent;
c) mixing the bio-oil and polar solvent during 5-120 minutes;
d) decanting the mixture of bio-oil and polar solvent;
e) retrieving the bio-oil fractions composition comprising anhydro sugars and acetic acid having a proportion of anhydro sugars to acetic acid from 0.8-1.5 in weight from one phase of the separated phases of the mixture from step d).

Accordingly, the process for the preparation of a bio-oil fractions composition comprising anhydro sugars and acetic acid having a proportion of anhydro sugars to acetic acid from 0.8-1.5 in weight uses a bio-oil in step a) that is in the form of an emulsion. Suitable bio-oils are typically obtained from the fast pyrolysis process. A fast pyrolysis process is adequate for converting lignocellulosic materials into bio-oil. The bio-oil comprises organic compounds from the thermal decomposition of lignocellulosic material, water, and some inorganic impurities. One example of process for obtaining bio-oil is described in the document WO2017201598.

Typically, the bio-oil of step a) is composed of:
organic acids such as ethanoic, propanoic and butanoic acids;
methoxyphenol isomers;
benzenediol isomers;
hydroxypropanone; and
levoglucosan anhydro sugars.

Further, the present process is optimal for bio-oil samples containing at least 20% of water, such that the bio-oil provided in step a) of the present process comprises at least 20% of a polar solvent, usually water or alcohol.

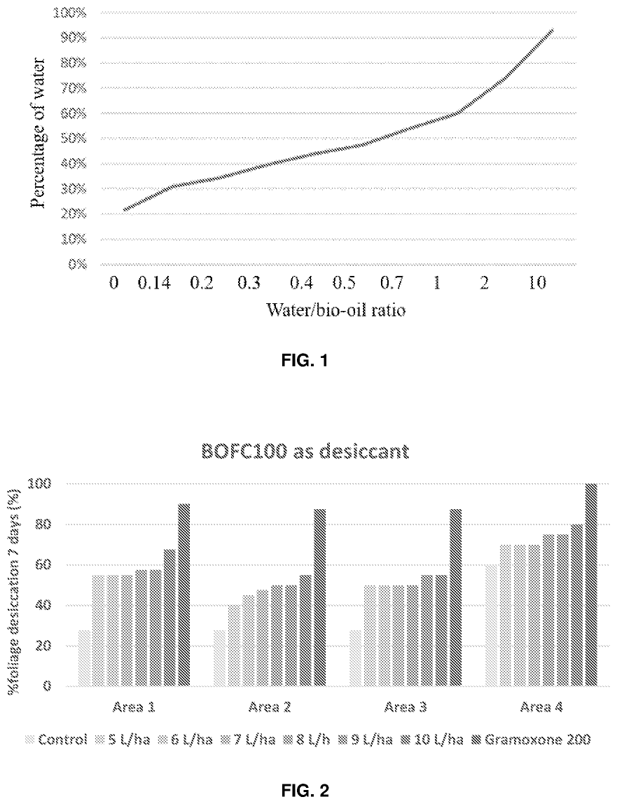

It should be noticed that the steps a), b) and c) may be performed in any order, i.e., the polar solvent is added to the bio-oil or the bio-oil is added to the polar solvent, mixing (step c) or with further mixing. Also, the agitation may start with the bio-oil or the polar solvent alone, while the other component is added. Preferably, the steps b) and c) are performed concurrently, i.e., the polar solvent is added to the bio-oil while mixture is performed. In this case, the polar solvent is added, for instance, the polar solvent may be added in an amount of 1% of the bio-oil provided in step a), while under mixture. Further amounts of polar solvent are then concordantly added until an optimum ratio of bio-oil to polar solvent is reached, i.e., another 1% of the mixture formed previously. FIG. 1 is a chart showing the relationship between a water/bio-oil ratio in a mixture of bio-oil and water and the percentage of water. Preferably, the polar solvent provided in step b) is added until a ratio from 0.1:1.0-0.5:1.0 of the polar solvent to the bio-oil provided in step a) is achieved; preferably the polar solvent is added until a ratio from 0.2:1.0-0.3:1.0 of the polar solvent to the bio-oil provided in step a) is achieved.

It should be noted that the bio-oil provided in step a) has some water content from the pyrolysis reaction. The polar solvent added in step b), that is, water, alcohol, an effluent from existing factories, their mixtures, or any other suitable polar solvent, interacts with the water in the bio-oil, changing the phase properties of the water-bio-oil system. As the original bio-oil emulsion breaks down, the organic phase is separated and start naturally decanting. In case water is used for bio-oil fractionation, a total amount from 30%-32% weight basis of water is necessary for the bio-oil phase separation.

In another embodiment, a total water content of less than 32% weight basis is satisfactory for the bio-oil phase separation. In another embodiment, a polar solvent, containing or not surfactants and salts, may be added at less than 30% weight basis for the bio-oil phase separation. For instance, bio-oil fractions compositions having from 0.1:1.0-0.5:1.0 polar solvent/bio-oil are preferred, which provide amounts of solvent adequate for easy handling during application, while also good separation of phases of the mixture.

The phase separation in oil and non-oil phase is usually achieved by decantation and/or centrifugation. In one embodiment, the phase decantation from step b) is performed in 3-48 hours. A centrifuge may be used after decantation to speed up the phase separation, or the decantation may be the only unit operation performed to carry out the phase separation. A decantation time of 3 hours is preferred, but longer periods are also adequate, up to 48 hours, when decantation is the only unit operation for separation. The residence time for centrifugation ranges from 1 minute to 25 minutes, preferably from 1 minute to 15 minutes. The rotation of the centrifuge ranges from 1,000 rpm to 8,000 rpm, preferably from 1,000 rpm to 5,000 rpm.

In another embodiment, the bio-oil and polar solvent are sent directly to the centrifuge, wherein no decantation is necessary before centrifugation. The residence time for centrifugation ranges from 1 minute to 15 minutes.

Once the non-oil phase is obtained, it forms a non-oil phase composition rich in anhydro sugars and acetic acid, usually having a proportion in weight of anhydro sugars to acetic acid from 0.8-1.5, comprising from 60-90% in weight of a polar solvent. Preferably, the polar solvent is selected from water, alcohol, effluents from existing factories, or mixtures thereof. Preferably, the alcohol is a C₁-C₆ alcohol. Even more preferably, the alcohol is methanol or ethanol.

The non-oil phase composition rich in anhydro sugars and acetic acid represents the bio-oil fractions composition and can be separated in a distillation process, wherein the resulting bottom phase contains levoglucosan (anhydro sugar) and the distillate phase contains acetic acid.

Usually, the bio-oil provided in step a) is usually an emulsion. To obtain a good phase separation after steps a), b) and c) of the process of the present invention, mixing the bio-oil with the polar solvent is preferably carried out at 100-600 rpm. The mixing rate is also related to the mass transfer between the phases.

In addition, low rotation causes early pyrolytic lignin lumps in the reactor, hence high agitation is preferred. Good separation is also achieved when the process is carried out at step c) under a temperature from 25° C.-60° C. High temperatures favor the solubilization of parts of the lignin in water, but this results in undesired properties in the application of the phases, as well as an unregular, less efficient phase separation. Usually, bio-oils are kept under 25° C. before use, so heating may be used to bring the bio-oil to at least 25° C. before use in step a). Alternatively, the heating may be applied to the bio-oil during step c) of the present process.

Achieving good separation should be understood as separation of the oil and non-oil phase, the latter being the aqueous phase based on a polar solvent, while the oil phase is commonly a lignin, also known as a pyrolytic lignin.

That is, the process of the present invention provides a bio-oil fractions composition as the aqueous phase from the fractionation of the bio-oil and a pyrolytic lignin as the oily phase from the fractionation of the bio-oil.

It is also provided an embodiment of the present invention that is a method for preparing a dispersion composed of an oil phase and a non-oil phase, by contacting a bio-oil with a polar solvent, the method comprising:
a) providing a bio-oil;
b) providing a polar solvent;
c) mixing the bio-oil and the polar solvent for 5-120 minutes.

Other embodiment of the invention refers to a method for extracting a bio-oil fractions composition from the dispersion, the method further comprising:
d) decanting the mixture of bio-oil and polar solvent for 3-48 hours and/or centrifuging the bio-oil and polar solvent mixture for 1-15 minutes;
e) retrieving the bio-oil fractions composition comprising anhydro sugars and acetic acid having a proportion of anhydro sugars to acetic acid from 0.8-1.5 in weight from one of the separated phases of the mixture from step d).

It is also provided a method for extracting a pyrolytic lignin from the dispersion, the method further comprising:
d) decanting the mixture of bio-oil and polar solvent for 3-48 hours and/or centrifuging the bio-oil and polar solvent mixture for 1-15 minutes;
e) retrieving the pyrolytic lignin from one of the separated phases of the mixture from step d).

In this case, the obtained lignin has a moisture content between 10 and 25% with a high acidity (pH 3-4) and high viscosity (>10,000 cP), provided with a molecular weight between 1,000-2,000 g/mol and solubility in acetone and alkaline solution.

It is also provided a pyrolytic lignin, which is the oily phase from the fractionation of the bio-oil, comprising from 15-20% phenols and from 12-16% benzaldehydes, preferably 20% phenols and 14% benzaldehydes. The pyrolytic lignin usually comprises less than 20% monomers having the remainder of the lignin composition polymerized in oligomers in an undefined structure, also with a low sulfur content, typically a sulfur content of less than 0.1%.

The pyrolytic lignin further comprises from 11-15% cyclic ketones and from 11-14% furfural.

In any case, the method for preparing said dispersion is achieved with an additional rate of the polar solvent at step b) from approximately 1%/min to approximately 5%/min of the total dispersion volume.

The method for preparing a dispersion or extracting a pyrolytic lignin may be carried out in a Batch Stirred Tank Reactor (BSTR) or in a continuous stirred-tank reactor (CSTR). Preferably, it is carried out in Batch Stirred Tank Reactor (BSTR) with a residence time from 1-120 min. A heat exchanger is present at the reactor, such as a heating or cooling jacket reactor or jacketed pipe reactor. Outer jackets are used for heating the reactor for desired temperatures higher than 25° C., preferably up to 60° C. In any case, the polar solvent may be added also to increase the temperature; for instance, heated water may be used in step b) at 25° C., 30° C., 35° C., 40° C., 45° C., 50° C., 55° C., 55° C., 60° C. or even higher temperatures. In any case, the reactor may use impellers such as turbine, helical, marine impellers or anchor impellers. Preferably, the impeller is a marine impeller at 100-600 rpm.

Anchor impellers are preferred to also remove any deposits that may be formed at the reactor walls while also provide good mixing.

It is also provided an embodiment of the present invention, wherein a salt is used as an additive during the bio-oil fractionation process. The concentration of the salt is up to 10% wt., based on the total weight of the bio-oil. Examples of suitable salts are potassium carbonate (K₂CO₃), sodium carbonate (Na₂CO₃), among others.

### EXAMPLES

The following examples will better illustrate this invention. The described particular features and ranges represent preferred but not limiting embodiments of this invention.

### Example 1—Bio-Oil Fractions Composition

For instance, a GC×GC Analysis—TOFMS (Two-Dimensional Gas Chromatography coupled with Time-of-flight mass spectrometry), which excludes oligomers and organic acids such as acetic acid, performed in exemplary bio-oil fractions compositions of the present invention, reveals ranges as depicted in Table 1.

TABLE 1

| Component | Range in the composition |
|---|---|
| Aldehydes | 5-15% |
| Anhydro sugars | 20-40% |
| Benzenediol isomers | 5-30% |
| Butyrolactone | 1-3% |
| Dimethoxyphenol isomers | 1% |
| Ketones | 3-6% |
| Esters | 2-8% |
| Phenolic compounds | 0.5-2% |
| Furaldehydes | 1-2% |
| Furandiones | 1-2% |
| Furanones | 5-10% |
| Pyranones | 0-0.5% |
| Hydroxyketones | 2-10% |
| Methoxybenzene isomers | 0.5% |
| Methoxybenzenediol | 1-2% |
| isomers | |
| Methoxyphenol isomers | 0.5% |
| Others | <10% |

### Example 2—Pyrolytic Lignin

For instance, a GC×GC Analysis—TOFMS (Two-Dimensional Gas Chromatography coupled with Time-of-flight mass spectrometry), excluding oligomers and organic acids such as acetic acid, performed in exemplary pyrolytic lignin obtainable by the present invention, reveals ranges as depicted in Table 2.

TABLE 2

| Component | Amount |
|---|---|
| Phenol, alkylphenol | 20.00% |
| Benzaldehydes | 14.00% |
| Cyclic Ketones | 13.90% |
| Furfural | 12.50% |
| Hydroxy aldehydes | 6.50% |
| Methoxy phenols | 3.70% |
| Dimethoxy Phenols | 2.70% |
| Others | 26.70% |

### Example 3—Bio-Oil Fractionation

### Example 3.1

This example relates to the bio-oil fractionation using process water as polar solvent at water/bio-oil ratio 0.2:1.0. The bio-oil had a water content of 36.07% weight basis, dynamic viscosity at 25° C. of 90.70 cP, pH 2.84 and elemental analysis as showed: carbon 37.20%, hydrogen 7.88%, nitrogen 0.06%, oxygen 54.85% and sulphur <0.1%. A jacketed reactor had a circulating cooling fluid at 25° C. Then a sample of 100 kg bio-oil was added to the reactor, and the impeller rotation was set to 200 rpm; in the next step, 20 kg of process water at 25° C. was added to the system. Agitation was maintained for 5 minutes. The bio-oil and solvent system laid to rest for 24 hours inside the reactor tank. Finally, the upper layer aqueous phase was pumped to an external tank, and the bottom layer oily phase was drained into a flask. Aqueous phase yield was 60% weight basis. Properties for aqueous and oily phases are presented in Table 3.

TABLE 3

| Sample | Aqueous phase | Oily phase |
|---|---|---|
| Water content (% weight) | 56.43 | 18.63 |
| Conductivity (mS/cm) | 1.29 | — |
| pH | 2.70 | — |
| Dynamic viscosity 25° C. (cP) | 3.15 | >10,000 |
| Acetic Acid (g/L) | 76.26 | — |
| Levoglucosan (g/L) | 78.83 | — |
| Ash Content (% weight) | — | 0.06% |
| Average molecular weight (Da) | — | 1007 |
| Weight average molecular weight | — | 2608 |
| (Da) | | |
| Elemental Analysis | — | Carbon 56.00% |
| | | Hydrogen 7.00% |
| | | Nitrogen 0.14% |
| | | Sulphur <0.1% |
| | | Oxygen 36.76% |

The aqueous phase presented a proportion of anhydrous sugars (levoglucosan) to acetic acid of 1.0 weight basis and solid content of 43.57%. The oily phase had a water content 48% lower than the bio-oil and a 50% increase in carbon content, configuring an adequate feedstock for fossil fuel replacement in combustion apparatus and refinery co-processing systems.

### Example 3.2

This example relates to the bio-oil fractionation using process water as polar solvent at water/bio-oil ratio 0.2:1.0. The bio-oil had a water content of 26.33% weight basis, dynamic viscosity at 25° C. of 91.50 cP, pH 3.22 and elemental analysis as showed: carbon 43.60%, hydrogen 7.89%, nitrogen 0.09%, oxygen 48.32% and sulphur <0.1%. A jacketed reactor had a circulating cooling fluid at 30° C. Then a sample of 100 kg bio-oil was added to the reactor, and the impeller rotation was set to 300 rpm; in the next step, 20 kg of process water at 25° C. was added to the system. Agitation was maintained for 5 minutes. The bio-oil and solvent system laid to rest for 24 hours inside the reactor tank. Finally, the upper layer aqueous phase was pumped to an external tank, and the bottom layer oily phase was drained into a flask. Aqueous phase yield was 60% weight basis. Properties for aqueous and oily phases are presented in Table 4.

TABLE 4

| Sample | Aqueous phase | Oily phase |
|---|---|---|
| Water content (% weight) | 54.87 | 19.99 |
| Conductivity (mS/cm) | 1.86 | — |
| pH | 3.20 | — |
| Dynamic viscosity 25° C. (cP) | 4.60 | >10,000 |
| Acetic Acid (g/L) | 62.83 | — |
| Levoglucosan (g/L) | 77.78 | — |
| Ash Content (% weight) | — | 0.10% |
| Average molecular weight (Da) | — | 757 |
| Weight average molecular weight | — | 1420 |
| (Da) | | |
| Elemental Analysis | — | Carbon 54.10% |
| | | Hydrogen 7.25% |
| | | Nitrogen 0.15% |
| | | Sulphur <0.1% |
| | | Oxygen 38.40% |

The aqueous phase presented a proportion of anhydrous sugars to acetic acid of 1.2 weight basis and solid content of 45.13%. The oily phase had a water content 24% lower than the bio-oil and a 24% increase in carbon content, configuring an adequate feedstock for fossil fuel replacement in combustion apparatus and refinery co-processing systems.

### Example 4—Centrifugation of the Oily Phase Resulting From the Bio-Oil Fractionation Process

This example relates to the bio-oil fractionation using process water as polar solvent at water/bio-oil ratio 1.0:1.0. The bio-oil had a water content of 36.07% weight basis, dynamic viscosity at 25° C. of 90.70 cP, pH 2.84 and elemental analysis as showed: carbon 37.20%, hydrogen 7.88%, nitrogen 0.06%, oxygen 54.85% and sulphur<0.1%. A glass reactor was immersed in a thermostatic bath fluid at 25° C. Then a sample of 1 kg bio-oil was added to the glass reactor, and the impeller rotation was set to 400 rpm; in the next step, 1 kg of process water at 25° C. was added to the system. Agitation was maintained for 5 minutes. The bio-oil and solvent system laid to rest for 24 hours inside the glass reactor. Finally, the upper layer aqueous phase was separated from the bottom layer oily phase. The oily phase was centrifugated to reduce its water content. Properties for aqueous and oily phases are presented in Table 5.

TABLE 5

| Sample | Aqueous phase | Oily phase |
|---|---|---|
| Water content (% weight) | 86.75 | 33.48 |
| Conductivity (mS/cm) | 1.82 | — |
| pH | 2.70 | — |
| Dynamic viscosity 25° C. (cP) | 1.60 | >10,000 |
| Acetic Acid (g/L) | 42.22 | — |
| Levoglucosan (g/L) | 42.02 | — |
| Ash Content (% weight) | — | 0.07% |
| Average molecular weight (Da) | — | 1013 |
| Weight average molecular weight | — | 2575 |
| (Da) | | |
| Elemental Analysis | — | Carbon 51.30% |
| | | Hydrogen 7.35% |
| | | Nitrogen 0.16% |
| | | Sulphur <0.1% |
| | | Oxygen 41.09% |

The aqueous phase presented a proportion of anhydrous sugars to acetic acid of 1.0 weight basis and solid content of 13.25%. Aqueous phase yield was 71% weight basis.

A sample of the oily phase was centrifugated in a pilot centrifugal spin dryer at 1160 rpm and for a residence time of 12 minutes. The final oily sample had a water content 17.88%, that is, the centrifugation resulted in a 47% water reduction compared to the original oily phase.

Also a factorial design for oily phase centrifugation was carried out in a bench scale centrifuge. Some experiments were performed varying the conditions of the centrifugation (the residence time and the rotation of the centrifuge), in order to analyze the results of reduction of water content in the oily phase. The results are presented in Table 6.

TABLE 6

| Experiment | Time | Rotation | Moisture |
|---|---|---|---|
| number | (minutes) | (rpm) | content (%) |
| 1 | 7.95 | 5000 | 22.68 |
| 2 | 22.05 | 5000 | 20.48 |
| 3 | 15.00 | 2180 | 21.46 |
| 4 | 15.00 | 7820 | 21.44 |
| 5 | 15.00 | 5000 | 21.31 |

The optimal condition for centrifugation at this scale was rotation of 5000 rpm and residence time of 22.05 minutes. The final oily sample had a water content 20.48%, that is, a 39% water reduction compared to the original oily phase resulting from a simple decantation.

### Example 5—Distillation of the Upper Layer Aqueous Phase Resulting From the Bio-Oil Fractionation Process

This example relates to the bio-oil fractionation using process water as polar solvent at water/bio-oil ratio 0.2:1.0. The bio-oil had a water content of 26.33% weight basis, dynamic viscosity at 25° C. of 91.50 cP, pH 3.22 and elemental analysis as showed: carbon 43.60%, hydrogen 7.89%, nitrogen 0.09%, oxygen 48.32% and sulphur <0.1%. A glass reactor was immersed in a thermostatic bath fluid at 40° C. Then a sample of 1 kg bio-oil was added to the glass reactor, and the impeller rotation was set to 400 rpm; in the next step, 1 kg of process water at 25° C. was added to the system. Agitation was maintained for 5 minutes. The bio-oil and solvent system laid to rest for 24 hours inside the glass reactor. Finally, the upper layer aqueous phase was separated from the bottom layer oily phase.

The aqueous phase (feed) was distilled in a bench scale apparatus. Boiler temperature was kept at 118° C. and this process was sustained until 50% of the aqueous phase was evaporated and collected as distillate. Finally, the aqueous phase distillation generated two samples: a bottom phase containing 156.64 g/L of levoglucosan and a distillate phase containing 105.42 g/L of acetic acid, which results in a weight proportion of anhydro sugar to acetic acid of about 1.5.

### Example 6—Applications of the Bio-Oil Fractions Composition

Exemplary applications of the bio-oil fractions composition obtained from the bio-oil are desiccants, bio-insecticide, polyurethane foams, resins, and also for the pyrolytic lignin obtained from the bio-oil.

For instance, the bio-oil fractions composition may be used as a desiccant herbicide in crops. One example is in a soybean crop, the pre-harvest desiccation stage with herbicides has some advantages such as: standardizing the soybean area, controlling weeds and bringing the harvest forward by an average of five to seven days. Soybean is usually desiccated with different herbicides, for instance Reglone (diquat) and Finale (glufosinate ammonium). Until 2020, banned Gramoxone 200 (paraquat) was extensively used for plant diseccation. The bio-oil fractions composition is an alternative to desiccate grains.

A bio-oil fractions composition, with about 2-3% acetic acid (approximately 20-30 g/L) was produced and it was diluted by 50%. Up to 10 L/ha of diluted bio-oil fractions composition was tested as desiccant, wherein the herbicide Gramoxone 200 was used as a reference.

FIG. 2 shows that the desiccation results obtained using 100% bio-oil fractions composition (BOFC100) were superior to the control (without desiccant), but inferior to Gramoxone considering 7 days of desiccation. Considering Paraquat based desiccants banning process worldwide, the bio-oil fractions composition, although less effective, has proven good performance as a desiccant and is a feasible and sustainable alternative.

The bio-oil fractions composition was also tested as desiccant in concentration of 3 liters per hectare and 7 liters per hectare, wherein the herbicides Finale (glufosinate-ammonium) and Reglone (diquat) were used as references.

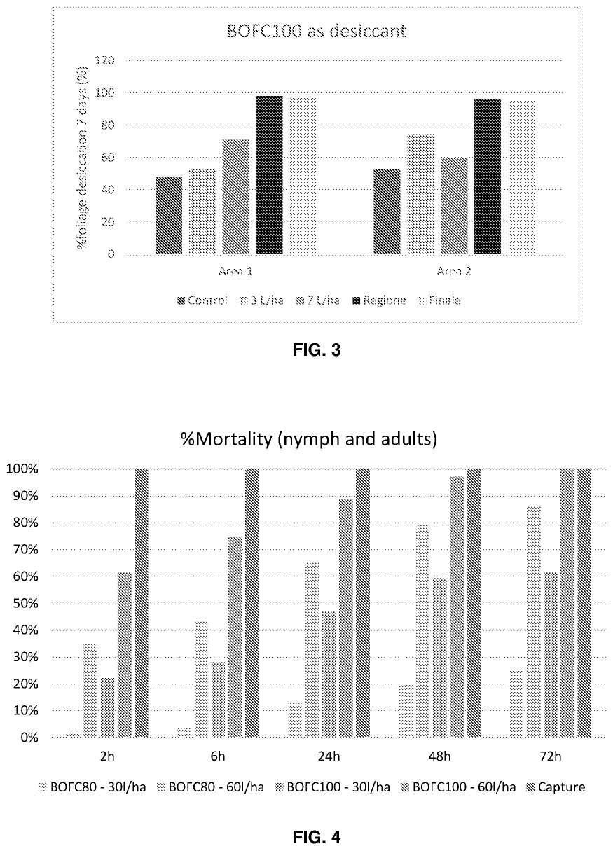

FIG. 3 shows that the desiccation results obtained using 100% bio-oil fractions composition (BOFC100) were superior to the control or untreated sample (without desiccant), but inferior to the commercial herbicides considering 7 days of soybean desiccation. That is, although less effective, the bio-oil fractions composition has proven good performance as a desiccant and is a feasible and sustainable alternative.

In this sense, it is also an object of the present invention the use of the bio-oil fractions composition as a desiccant in crops, as well as a desiccant herbicide comprising the bio-oil fractions composition.

The use of the bio-oil fractions composition as a crop insecticide is also of interest in the present invention. For instance, the bio-oil fractions composition was assessed as a bio-insecticide to control the bronze bug (PVB—Thaumastocoris peregrinus). FIG. 4 shows the use of the bio-oil fractions composition (BOFC) in different aqueous concentrations (80% and 100%) and compared with the insecticide Capture with volume applications of 30 L/ha and 60 L/ha.

Although not as aggressive as the Capture insecticide, the bio-oil fractions composition has proven insecticide capabilities, especially when used at 80% and 100% (BOFC80 and BOFC100) at 60 L/ha. In this sense, it is also an object of the present invention the use of the bio-oil fractions composition as a crop insecticide, as well as a crop insecticide comprising the bio-oil fractions composition.

The bio-oil fractions composition (BOFC) may also be used in other different aqueous concentrations, particularly, 20%, 40% and 60% as a bio-insecticide to control the bronze bug (PVB—Thaumastocoris peregrinus).

### Example 7—Use of a Salt as an Additive During the Bio-Oil Fractionation

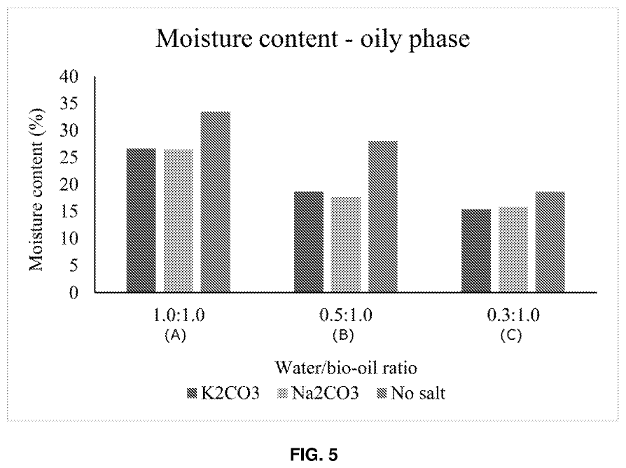

This example relates to the bio-oil fractionation using process water as polar solvent, potassium carbonate (K₂CO₃) and sodium carbonate (Na₂CO₃) as salts for salting out (10% weight basis). The moisture content (%) of the oily phase measured for water/bio-oil ratios of 1.0:1.0 (A), 0.5:1.0 (B) and 0.3:1.0 (C) are illustrated in FIG. 5.

The bio-oil had a water content of 26.33% weight basis, dynamic viscosity at 25° C. of 91.50 cP, pH 3.22 and elemental analysis as showed: carbon 43.60%, hydrogen 7.89%, nitrogen 0.09%, oxygen 48.32% and sulphur <0.1%. A glass reactor was immersed in a thermostatic bath fluid at 40° C. Then a sample of 1 kg bio-oil was added to the glass reactor, and the impeller rotation was set to 400 rpm; in the next step, 1 kg of process water at 25° C. was added to the system. Agitation was maintained for 5 minutes. The bio-oil and solvent system laid to rest for 24 hours inside the glass reactor. Finally, the upper layer aqueous phase was separated from the bottom layer oily phase.

In this example, when the salt is used as an additive, the solubility of the solvent molecules in the aqueous phase decreases, leading to the formation of a two-phase system. The addition of salt is also used to facilitate the removal of water from the organic medium, as the ionic strength of the salt makes the aqueous phase less compatible with the organic phase.

It is understood that where a parameter range is provided, all integers and ranges within that range, and tenths and hundredths thereof, are also provided by the embodiments. For example, “5-10%” includes 5%, 6%, 7%, 8%, 9%, and 10%; 5.0%, 5.1%, 5.2% . . . 9.8%, 9.9%, and 10.0%; and 5.00%, 5.01%, 5.02% . . . 9.98%, 9.99%, and 10.00%, as well as, for example, 6-9%, 5.1%-9.9%, and 5.01%-9.99%. Similarly, where a list is presented, unless stated otherwise, it is to be understood that each individual element of that list, and every combination of components of that list, is a separate embodiment. For example, “1, 2, 3, 4, and 5” encompasses, among numerous embodiments, 1; 2; 3; 1 and 2; 3 and 5; 1, 3, and 5; and 1, 2, 4, and 5.

The above embodiments are to be understood as illustrative examples of the invention. Further embodiments of the invention are envisaged. It is to be understood that any feature described in relation to any one embodiment may be used alone, or in combination with other features described, and may also be used in combination with one or more features of any other of the embodiments, or any combination of any other of the embodiments. Furthermore, equivalents and modifications not described above may also be employed without departing from the scope of the invention, which is defined in the accompanying claims.

1. A bio-oil fractions composition, comprising anhydro sugars and acetic acid wherein a weight proportion of anhydro sugars to acetic acid is from 0.8-1.5.

2. The bio-oil fractions composition of claim 1, wherein the anhydro sugars are present from 50 g/L-110 g/L and the acetic acid is present from 70 g/L-120 g/L.

3. The bio-oil fractions composition of claim 1, having a pH from 3-4 and a dynamic viscosity from 2-5 cP.

4. The bio-oil fractions composition of claim 1, comprising from 1-2% of hydroxyacetones, from 1-3% of glycolaldehydes and/or from 4-6% of benzenediols.

5. The bio-oil fractions composition of claim 1, wherein the proportion of anhydro sugars to acetic acid is 1.

6. The bio-oil fractions composition of claim 1, wherein the anhydro sugars are levoglucosan.

7. The bio-oil fractions composition of claim 4, wherein the benzenediols comprise 1,4-benzenediol, 1,3-benzenediol and 1,2-benzenediol.

8. The bio-oil fractions composition according to claim 1, wherein the bio-oil fractions composition comprises from 60-90% wt. of polar solvent.

9. The bio-oil fractions composition according to claim 8, wherein the polar solvent is selected from the group consisting of water, alcohol, effluent from existing factories and mixtures thereof.

10. The bio-oil fractions composition according to claim 9, wherein the polar solvent is an alcohol, wherein the alcohol is a C₁-C₆ alcohol, preferably methanol or ethanol.

11. The bio-oil fractions composition according to claim 1, wherein the bio-oil fractions composition further comprises a component selected from the group consisting of: a) surfactant; and b) salt.

12. The bio-oil fractions composition according to claim 11, wherein the surfactant is selected from the group consisting of alkyl benzene sulphonates, methyl methacrylate, kraft lignin, lignosulphonates, cellulose derivatives and mixtures thereof.

13. The bio-oil fractions composition according to claim 11, wherein the salt is selected from the group consisting of sodium chloride, calcium chloride, magnesium chloride, phosphates, pyrophosphates, sulfates, sulfides, carbonates, nitrates salts and mixtures thereof.

14. A pyrolytic lignin comprising from 15-20% phenols and from 12-16% benzaldehydes.

15. The pyrolytic lignin according to claim 14, further comprising from 11-15% of cyclic ketones and from 11-14% of furfural.

16. The pyrolytic lignin of claim 14, further comprising a low sulfur content of less than 0.1%.

17. (canceled)

18. A process for the preparation of a bio-oil fractions composition comprising anhydro sugars and acetic acid having a proportion of anhydro sugars to acetic acid from 0.8-1.5 in weight comprising:
a) providing a bio-oil;
b) providing a polar solvent;
c) mixing the bio-oil and the polar solvent during 5-120 minutes;
d) decanting the mixture of bio-oil and polar solvent and/or centrifuging the bio-oil and polar solvent mixture;
e) retrieving the bio-oil fractions composition comprising anhydro sugars and acetic acid having a proportion of anhydro sugars to acetic acid from 0.8-1.5 in weight from one phase of the separated phases of the mixture from step d).

19. The process of claim 18, wherein the bio-oil is a bio-oil emulsion.

20.-21. (canceled)

22. The process of claim 18, wherein the polar solvent provided in step b) forms a ratio from 0.1:1.0-0.5:1.0 of the polar solvent to the bio-oil provided in step a).

23.-34. (canceled)

35. The bio-oil fractions composition of claim 1, wherein the bio-oil fractions composition comprises a desiccant herbicide composition or a crop insecticide composition.

36.-37. (canceled)
